(19) United States
(12) Patent Application Publication
(10) Pub. No.: US 2023/0320955 A1
(43) Pub. Date: Oct. 12, 2023

(54) TRANSPARENT LIQUID HAIR MASK WITH TRANSFORMING APPEARANCE FOR A LIGHTWEIGHT APPLICATION

(71) Applicant: Henkel AG & Co. KGaA, Duesseldorf (DE)

(72) Inventors: Tina AHNFELDT, Strukdorf (DE); Jasmin INGWERSEN, Hamburg (DE); Sylvia KERL, Boenningstedt (DE)

(73) Assignee: Henkel AG & Co. KGaA, Duesseldorf (DE)

(21) Appl. No.: 18/044,183

(22) PCT Filed: Aug. 5, 2021
(86) PCT No.: PCT/EP2021/071843

§ 371 Date: Mar. 6, 2023

(30) Foreign Application Priority Data
Sep. 7, 2020 (DE) 10 2020 123 258.6

(51) Int. Cl. A61K 8/34; A61K 8/86; A61K 8/41; A61Q 5/00

(52) U.S. Cl. CPC A61K 8/345; A61K 8/86; A61K 2800/262; A61Q 5/002; A61K 8/416

(57) ABSTRACT

A hair care composition is disclosed. The hair care composition is formulated to present as a single-phase liquid, typically transparent and clear, and comprises at least one polyhydric alcohol at least one polyethylene glycol, at least one monohydric alcohol, and at least one quaternary ammonium salt. A method of treating hair (e.g. human hair) is also disclosed. The method includes contacting the hair with the hair care composition for an exposure time (e.g. from about 1 second to about 3 minutes), and then rinsing the hair (e.g. with water) to stop or reduce the contact and exposure of the hair to the hair care composition. The method may include working in and/or massaging the composition into the hair during the exposure time to enhance the contacting.

### CROSS-REFERENCE TO RELATED APPLICATION

This application is a U.S. National-Stage entry under 35 U.S.C. § 371 based on International Application No. PCT/EP2021/071843, filed Aug. 5, 2021, which was published under PCT Article 21(2) and which claims priority to German Application No. 102020123258.6, filed Sep. 7, 2020, which are all hereby incorporated in their entirety by reference.

### TECHNICAL FIELD

Personal hygiene plays an important role in the satisfaction and well-being of people. There are several reasons for this. On the one hand, there is a widespread social consensus that a well-groomed appearance makes interaction with one another more pleasant and can also be seen as a sign of respect towards fellow human beings. On the other hand, many people also feel more comfortable for intrinsic reasons when they are well-groomed. However, even the care itself is for many a pleasant relaxing and perhaps somewhat meditative experience associated with pleasant feelings. In this respect, personal hygiene also has considerable emotional and playful aspects.

Hair care often plays a central role in the overall body care. Healthy shiny, but not greasy hair with resilience and volume is widely seen as a special sign of beauty but also health. At the same time, however, the widespread modern lifestyle can significantly affect long hair in particular. Frequent washing and blow-drying can leave traces as well as hard water, dry air, styling products or tints and dyes, which then results in a special need for care and/or repair of the hair.

### BACKGROUND

Significant care and repair effects are achieved with hair masks or hair treatments, which can be applied to wet hair after cleaning with water and shampoo but before conditioning. Such products are usually emulsions that contain nourishing ingredients that can penetrate the hair structure to some extent, but this requires some exposure time.

Maintaining the correct exposure time can be challenging or uncomfortable for users and can detract from the positive experience. Too short an exposure time results in only a slight conditioning effect, while too long an exposure time can lead to various undesirable effects, depending on the product, from greasy-looking hair to hair breakage. In addition, if the exposure time is waited for in the shower, for example, this can result in higher water consumption, which, due to the treatment of groundwater or surface water to drinking water, is associated with technical effort and energy consumption and, of course, also with a cost burden for the consumer. In areas where water resources are scarce, increased water use may also be less desirable for other reasons.

Apart from this, emulsion-based products have for some time been increasingly perceived as somewhat old-fashioned, and a steadily growing group of users wants products that are clear, transparent, thin-bodied, and possibly even colorless. These are rated as light, modern, and little or no discomfort, and address the emotional playful aspect of personal hygiene mentioned at the beginning of this article.

The prevailing opinion in the technical field, however, is that such a product appearance and the strong care properties required for a hair mask cannot be reconciled, especially if even a short contact time is to be sufficient to achieve them, or even if it is to be achieved that no separate contact time is to be observed at all and that the contact time alone, which occurs when a product is distributed in the hair, is to be sufficient to achieve the intended care effect.

### BRIEF SUMMARY

A hair care composition is provided. The hair care composition is formulated to present as a single-phase liquid, typically transparent and clear, and comprises at least one polyhydric alcohol at least one polyethylene glycol, at least one monohydric alcohol, and at least one quaternary ammonium salt.

A method of treating hair (e.g. human hair) is also provided. The method includes contacting wet, damp, or moist hair with the hair care composition for an exposure time (e.g. from about 1 second to about 3 minutes), and then rinsing the hair (e.g. with water) to stop or reduce the contact and exposure of the hair to the hair care composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The inventors have set themselves the task of developing a transparent hair mask that offers superior care and repair effect even with only a very short or even no separate exposure time.

This task is solved by the formulations, methods of use thereof and uses described below, by which the inventors have been able to provide a clear liquid hair mask with transformative appearance for non-weighing care. The term “transforming appearance” is to be understood as the hair mask significantly changes its appearance when brought into contact with wet hair, as described in more detail below. The term “non-complaining” is to be understood as meaning that no complaining effect is perceived after application.

More specifically, the present disclosure provides a hair care composition comprising: at least one polyhydric alcohol, at least one polyethylene glycol, at least one monohydric alcohol, and at least one quaternary ammonium salt.

With such a formulation, it is possible to keep the conditioning ingredients in solution, creating a clear transparent product. A drying effect occasionally attributed to alcohols is not observed, as only an extremely short exposure time is required, which is presumably due to the fact that the alcohols are also helpful for rapid penetration of the conditioning substances into the hair structure. Another very positive effect was noted. Due to the fact that emulsification of the mask takes place only on wet hair, a very fine creamy foamy consistency and a slight warming is created, which was consistently rated very positively by test persons.

Thus, because the hair care compositions contain essential ingredients as a solution rather than, for example, as an emulsion or suspension, they can be clear and transparent, unlike conventional emulsion-based products, and it is preferred that they be clear and transparent. This should be understood here to mean that something described as “clear and transparent,” such as hair care compositions, essentially does not scatter light in the range usually visible to humans.

In addition, a comparatively low viscosity of hair care compositions compared to conventional products is a consequence of the formulation just described. In addition to practical advantages, for example the fact that such a hair care composition can be easily removed or emptied from a package with little residue, there is again the perceptual and emotional aspect. Low viscosity products are perceived as light and modern. Preferably, the viscosity of the hair care compositions as contemplated herein is at most about 500 mPas, preferably at most about 350 mPas and in particular preferably at most about 200 mPas when measured with a rotational viscometer, model VT 550, from Haake, as detailed in the enclosed instructions and briefly described below. For viscosity measurement, a sample is measured in the gap between two coaxial cylinders. The inner cylinder (rotor) is rotated against the outer, fixed cylinder (stator), creating laminar flow in the fluid. When a certain rotational speed is applied, a defined shear rate is generated in the gap between the cylinders, the torque is measured at the rotor and converted into shear stress.

Furthermore, the present disclosure provides a hair care composition as previously described, wherein the at least one polyhydric alcohol is a C3 or C4 alcohol or a mixture thereof, preferably glycerol, propanediol or a mixture thereof and more preferably the mixture of glycerol and propanediol and/or is present in a concentration of from about 15 to 45 wt. %, preferably from about 21 to about 42 wt. % and more preferably from about 25 to about 40 wt. %, each based on the total weight of the hair care composition therein.

These polyhydric alcohols serve as humectants, among other things. Their goal is to provide skin and hair with optimal moisture. They keep the skin healthy and elastic, prevent the premature formation of wrinkles and strengthen the natural skin barrier. After all, this barrier function is the most important task of the skin, it protects the organism from drying out and from harmful external influences. However, the humectants also directly improve the suppleness of skin and hair. In the context of the formulations disclosed herein, the concentration ranges described have been shown to be particularly suitable for producing the desired effect described to the extent intended without producing significant undesirable effects or unduly restricting the freedom of formulation as far as the other ingredients are concerned.

Furthermore, the present disclosure provides a hair care composition as described above, wherein the at least one polyethylene glycol is a polyethylene glycol having a molecular weight of from about 200 to about 600 and preferably the polyethylene glycol known under the INCI designation PEG-8 having a molecular weight of about 400 and/or is present in a concentration of from about 15 wt. % to about 45 wt. %, preferably from about 21 wt. % to about 42 wt. % and more preferably from about from 25 wt. % to about 40 wt. %, in each case based on the total weight of the hair care composition therein.

Such polyethylene glycol has a beneficial effect for the in-situ formation of an emulsion on the hair and scalp desired in the context of the present disclosure and, by extension, also has a somewhat refatting effect. The concentration ranges described have been found to be particularly suitable in the context of the formulations disclosed herein to produce the desired effect described to the extent intended without producing significant undesirable effects or unduly restricting the freedom of formulation with respect to the other ingredients.

Further provided by the present disclosure is a hair care composition as previously described, wherein the at least one monohydric alcohol is ethanol, propanol, butanol or a mixture thereof, preferably ethanol, propanol or a mixture thereof and more preferably ethanol and/or is present in a concentration of from about 15 wt. % to about 45 wt. %, preferably from about 21 wt. % to about 42 wt. % and more preferably from about 25 wt. % to about 40 wt. %, each based on the total weight of the hair care composition therein.

These alcohols have good properties for dissolving the skin care ingredients, are not absorbed by the skin in significant quantities during the short exposure time and are easy to produce from renewable raw materials. In the context of the formulations disclosed herein, the concentration ranges described have been shown to be particularly suitable for producing the desired effect described to the extent intended without producing significant undesirable effects or unduly restricting the freedom of formulation as far as the other ingredients are concerned.

Further provided by the present disclosure is a hair care composition as described above, wherein the at least one quaternary ammonium salt is an alkyl trimethylammonium salt, preferably a C14 to C18 alkyl trimethylammonium salt and more preferably hexadecyltrimethylammonium chloride, also known as cetrimonium chloride, and/or is present in a concentration of from about 0.1 wt. % to % to about 12 wt. %, preferably from about 0.7 wt. % to about 10 wt. % and more preferably from about 1 wt. % to about 9 wt. %, each based on the total weight of the hair care composition.

Such a quaternary ammonium salt reseals the opened cuticle after shampooing, making the hair less sensitive again. It then loses no moisture, is protected from drying out and achieves a new shine and suppleness without perceiving a weighed-down feeling. In the context of the formulations disclosed herein, the concentration ranges described have been shown to be particularly suitable for producing the desired effect described to the extent intended without producing significant undesirable effects or unduly restricting the freedom of formulation as far as the other ingredients are concerned.

The previously mentioned quaternary ammonium salts have proven to be particularly suitable. In principle, however, other cationic surfactants suitable for use on the human body are also suitable. These are exemplified by at least one water-solubilizing, cationic group, such as a quaternary ammonium group, or by at least one water-solubilizing, cationizable group, such as an amine group and furthermore at least one lipophilic alkyl group with about 6 to 30 C atoms, or also by at least one imidazole group or at least one imidazylalkyl group.

Suitable are, for example, quaternary ammonium compounds having at least one C8-C24 alkyl radical, esterquats and amidoamines each having at least one C8-C24 acyl radical, and mixtures thereof. Preferred quaternary ammonium compounds having at least one C8-C24 alkyl radical are ammonium halides, in particular chlorides, and ammonium alkyl sulfates, such as methosulfates or ethosulfates, such as C8-C24 alkyltrimethylammonium chlorides, C8-C24 dialkyldimethylammonium chlorides and C8-C24 trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI designations quaternium-27, quaternium-83, quaternium-87 and quaternium-91. The alkyl chains of the surfactants mentioned above preferably have 8 to 24 carbon atoms. Esterquats are cationic surfactants that contain both at least one ester function and at least one quaternary ammonium group as a structural element and furthermore at least one C8-C24 alkyl radical or C8-C24 acyl radical. Preferred esterquats are quaternized ester salts of fatty acids with triethanolamine, quaternized ester salts of fatty acids with diethanolalkylamines and quaternized ester salts of fatty acids with 1.2-dihydroxypropyl dialkylamines. Such products are sold under the trademarks Stepantex®, Dehyquart® and Armocare®. N,N-Bis(2-palmitoyloxyethyl) dimethylammonium chloride, distearoylethyl dimonium methosulfate and distearoylethyl hydroxyethylmonium methosulfate are preferred examples of such esterquats.

Furthermore, the present disclosure provides a hair care composition as previously described, in which further water is present therein in a concentration of less than about 20 wt. %, preferably less than about 15 wt. %, and more preferably less than about 10 wt. %, in each case based on the total weight of the hair care composition.

There are advantages for formulability in accepting a certain water content, since certain commercially available precursors usually contain certain amounts of water, but there are also advantages for distributability and action on the hair, since the volume is increased somewhat and the concentrations of active ingredients do not become too high. If the water content is kept low enough so as not to impair the desired effect that emulsification only occurs on the hair, then a corresponding procedure is practically without disadvantages. In the context of the formulations disclosed herein, the concentration ranges described above have been found to be particularly suitable for producing the desired effect described to the extent intended without producing significant undesirable effects or unduly restricting the freedom of formulation as far as the other ingredients are concerned.

Further, the present disclosure provides a hair care composition as previously described, wherein it comprises, as the at least one polyhydric alcohol, the at least one polyethylene glycol, the at least one monohydric alcohol and the at least one quaternary ammonium salt, glycerin, the polyethylene glycol known by the INCI designation PEG-8, ethanol, and hexadecyltrimethylammonium chloride. Such a combination has been found to be particularly advantageous for achieving the beneficial effects of the present disclosure already described.

Further provided by the present disclosure is a hair care composition as previously described, wherein it comprises from about 25 to about 35 wt. % of at least one polyhydric alcohol, from about 25 to about 35 wt. % of at least one polyethylene glycol, from about 25 to about 35 wt. % of at least one monohydric alcohol, and from about 6 to about 10 wt. % of at least one quaternary ammonium salt, each based on the total weight of the hair care composition. These concentration ranges combined have been found to be particularly advantageous for achieving the beneficial effects of the present disclosure already described.

Silicones are also widely used as emollients in generic hair care products. This is often considered to be largely without alternative for achieving the desired care and repair effect. The hair care compositions as contemplated herein achieve outstanding care and repair effects even without silicones. However, they are fully compatible with silicones and may contain silicones if desired in certain cases.

In general, at least the hair care compositions as contemplated herein make it possible to dispense with organosilicon compounds, in particular silicones, in these very hair care compositions. In the context of the present disclosure, “silicone-free” or “free of silicones” is understood to mean that what is described as “silicone-free” or “free of silicones”, for example, the hair care compositions described herein, is free of organosilicon compounds, in particular free of silicones, trisiloxanes and silicone oils.

Further provided by the present disclosure is a method comprising the steps of: bringing the hair care composition as previously described into contact with preferably wet or damp hair of a human or animal, in particular long scalp hair of a human, optionally incorporating and/or massaging to enhance the contact with the hair, optionally waiting for a certain exposure time in the range of from about 1 second to 3 minutes, preferably from about 2 seconds to about 1 minute and more preferably from about 5 to about 10 seconds, and rinsing the hair, preferably with an aqueous liquid, more preferably with water, to terminate or at least reduce further exposure to the hair care composition.

Through such a process, the previously described advantages are actually realized and can be experienced. In particular, it should be emphasized that this offers a new experience in the form of emulsification on the hair with the pleasant sensations already described, and that the only very short application time required is usually already automatic and hardly needs to be observed separately, which makes the application simple, pleasant and less prone to errors.

Further, the present disclosure provides a use of the hair care composition as previously described for caring for preferably wet or damp hair of a human or animal, in particular long scalp hair of a human.

Through such use, the previously described advantages are actually realized and experienced. In particular, it should be emphasized that this offers a new experience in the form of emulsification on the hair with the pleasant sensations already described, and that the only very short application time required is usually already automatic and hardly needs to be observed separately, which makes the application simple, pleasant and less prone to errors.

### Other Ingredients

Other ingredients may be present in the hair care compositions of the present disclosure, some of which are described below. If there are overlaps with the ingredients discussed in detail above, the ingredients discussed later are also included, unless this is explicitly described otherwise at the respective point.

In a preferred embodiment, hair care compositions as contemplated herein contain at least one hair-conditioning active ingredient selected from the group of
cationic polymers, preferably cationic polymers of natural origin and/or
humectants and/or
oils, preferably natural (vegetable) oils and/or
Trimethylglycine and/or
Vitamins and/or
Plant extracts and/or
Protein hydrolysates.

Suitable cationic polymers of natural origin within the meaning of the present disclosure are understood to mean, for example:
quaternized cellulose derivatives, such as those commercially available under the names Celquat® and Polymer JR®,
hydrophobically modified cellulose derivatives, for example the cationic polymers marketed under the trade name SoftCat®,
cationic alkyl polyglycosides,
cationised honey, for example the commercial product Honeyquat® 50,
cationic guar derivatives, such as in particular the products marketed under the trade names Cosmedia®Guar, N-Hance® and Jaguar®.
cationic cassia and/or inulin derivatives such as the cationic polymers known under the INCI designations Cassia Hydroxypropyltrimonium Chloride and Hydroxypropyltrimonium Inulin.

Quaternized cellulose polymers, hydrophobically modified cationic cellulose derivatives and/or cationic guar derivatives are preferred.

Particularly preferred are the cationic polymers known under the INCI designations guar hydroxypropyltrimonium chloride, polyquaternium-10, polyquaternium-67; guar hydroxypropyltrimonium chloride is particularly preferred.

The hair care compositions as contemplated herein may contain cationic polymers—preferably guar hydroxypropyltrimonium chloride—preferably in a proportion by weight of from about 0.01 to about 2 wt. %, more preferably from about 0.02 to about 1.5 wt. % and in particular from about 0.03 to about 1 wt. % of the total weight of the hair care composition.

Suitable humectants within the meaning of the present disclosure are preferably fiber structure-improving active ingredients such as polyols (i.e., physiologically compatible polyalcohols or polyhydroxyl compounds) with 2 to 20 carbon atoms and at least two hydroxyl groups. Typical examples are straight-chain, branched or cyclic alkanols with 2 to 10, preferably with 2 to 6 carbon atoms, such as glycerol, 1,2-propanediol and/or diglycerol, glycols such as polyethylene glycols and/or polypropylene glycols, and sugar alcohols or sugar derivatives such as fructose, glucose, lactose, galactose, sucrose, sorbitol, mannitol, etc. Glycerol is preferred.

The concentrations in which essential representatives of these humectants are present in the hair care compositions as contemplated herein have already been addressed. If additional humectants are present from those mentioned here, for which concentrations have not yet been mentioned, these are present in addition to those previously thematized, preferably in a proportion by weight of from about 0.05 to about 5 wt. %, more preferably from about 0.1 to about 4 wt. % and in particular from about 0.25 to about 3 wt. % of the total weight of the hair care composition.

For the purposes of the present disclosure, “natural” oils are preferably understood to mean a vegetable oil, and more preferably a cold-pressed vegetable oil.

“Cold-pressed oil” means an oil obtained by cold pressing and/or cold extraction of the respective plant (or part of the plant) at a temperature of about 40° C. or less, preferably about 35° C. or less, and more preferably about 30° C. or less.

Preferred oils that can be obtained by cold pressing or cold extraction and are suitable for use in the compositions as contemplated herein are triglycerides and mixtures of triglycerides such as coconut oil, (sweet) almond oil, walnut oil, peach kernel oil, apricot kernel oil, avocado oil, tea tree oil, soybean oil, sesame oil, Sunflower oil, Tsubaki oil, Evening primrose oil, Rice bran oil, Palm kernel oil, Mango kernel oil, Meadow foam oil, Safflower oil, Macadamia nut oil, Grape seed oil, Amaranth seed oil, Argan oil, bamboo oil, olive oil, wheat germ oil, pumpkin seed oil, mallow oil, hazelnut oil, safflower oil, canola oil, sasanqua oil, jojoba oil, rambutan oil, cocoa butter and shea butter are used.

The cold-pressed or cold-extracted oil is preferably present in dispersed form in the hair care compositions of the present disclosure, wherein the droplet size of the dispersed oil is about 0.1 to about 5 μm, preferably from about 0.5 to about 3 μm.

The hair care compositions as contemplated herein contain natural oils—preferably cold-pressed natural oils—preferably in a proportion by weight of from about 0.01 to 2 wt. %, more preferably from about 0.025 to about 1.5 wt. % and in particular from about 0.05 to about 1 wt. % of the total weight of the hair care composition.

Trimethylglycine can preferably be used in the hair care compositions as contemplated herein in a proportion by weight of from about 0.1 to about 3 wt. %, more preferably from about 0.25 to about 2.5 wt. % and in particular from about 0.5 to about 2 wt. % of the total weight of the hair care composition.

Suitable “vitamins” are preferably the following vitamins, provitamins and vitamin precursors and their derivatives:
Vitamin A: the group of substances known as vitamin A includes retinol (vitamin A₁) and 3,4-didehydroretinol (vitamin A₂). The b-carotene is the provitamin of retinol. Vitamin A components include vitamin A acid and its esters, vitamin A aldehyde and vitamin A alcohol and its esters such as palmitate and acetate.
Vitamin B: the vitamin B group or complex includes, among others.
Vitamin B₁ (Thiamine)
Vitamin B₂ (Riboflavin)
Vitamin B₃. The compounds nicotinic acid and nicotinamide (niacinamide) are frequently listed under this designation.
Vitamin B₅ (pantothenic acid and panthenol). Within this group, panthenol is preferred. Applicable derivatives of panthenol are especially the esters and ethers of panthenol, pantolactone and cationically derivatized panthenols. Individual representatives include panthenol triacetate, panthenol monoethyl ether and its monoacetate as well as cationic panthenol derivatives.
Vitamin B₆ (pyridoxine as well as pyridoxamine and pyridoxal).
Vitamin C (ascorbic acid): use in the form of palmitic acid ester, glucosides or phosphates may be preferred. Use in combination with tocopherols may also be preferred.
Vitamin E (tocopherols, especially a-tocopherol).
Vitamin F: the term “vitamin F” usually refers to essential fatty acids, especially linoleic acid, linolenic acid and arachidonic acid.
Vitamin H: Vitamin H is the compound (3aS,4S,6aR)-2-oxohexahydrothienol[3,4-d]-imidazole-4-valeric acid, for which the trivial name biotin has since become established.

Especially preferred are vitamins, provitamins and vitamin precursors from the groups A, B, E and H. Nicotinamide, biotin, pantolactone and/or panthenol are particularly preferred; nicotinamide and/or panthenol are especially preferred.

The proportion by weight of the vitamin(s), vitamin derivative(s), and/or vitamin precursor(s) in the total weight of the cosmetic hair care compositions as contemplated herein is preferably from about 0.005 to about 2 wt. about particularly preferably from about 0.008 to about 1 wt. % and in particular about 0.01 to about 0.50 wt. %.

Suitable plant extracts are mainly the extracts of green tea, oak bark, nettle, witch hazel, hops, henna, chamomile, burdock root, horsetail, hawthorn, linden blossom, almond, Aloe vera, spruce needle, horse chestnut, date palm, cinnamon tree, sandalwood, juniper, coconut, mango, apricot, lime, wheat, kiwi, melon, orange, grapefruit, sage, rosemary, birch, mallow, Valerian, Meadowfoam, Quender, Yarrow, Thyme, Melissa, Hauhechel, Coltsfoot, Marshmallow, Meristem, Ginseng, Coffee, Cocoa, Moringa, Ginger Root and Ayurvedic plant extracts such as Aegle Marmelos (Bilwa), Cyperus Rotundus (Nagar Motha), Emblica Officinalis (Amalki), Morida Citrifolia (Ashyuka), Tinospora Cordifolia (Guduchi), Santalum album, (Chandana), Crocus Sativus (Kumkuma), Cinnamonum Zeylanicum and Nelumbo Nucifera (Kamala), sweet grasses such as wheat, barley, rye, oats, spelt, corn, the different varieties of millet (panicle millet, Finger millet, foxtail millet as examples), sugarcane, ryegrass, meadow foxtail, smooth oats, bunchgrass, meadow fescue, pipegrass, bamboo, cotton grass, lampbush grasses, Andropogonodeae (Imperata Cylindrica also called flame grass or cogon grass), buffalo grass, silt grasses, dogtooth grasses, love grasses, Cymbopogon (lemon grass), Oryzeae (rice), Zizania (wild rice), Beach oats, perennial oats, honey grasses, quaking grasses, meadow grasses, couch grasses, and Echinacea, especially Echinacea angustifolia DC, Echinacea paradoxa (Norton), Echinacea simulata, E. atrorubens, E. tennesiensis, Echinacea strigosa (Mc Gregor), Echinacea laevigata, Echinacea purpurea (L.) Moench and Echinacea pallida (Nutt), all kinds of seaweeds, coral moss and seaweed (such as Macrocystis Pyrifera extract), all kinds of vines and pericarp of Litchie chinensis.

Suitable extracts can be obtained from the fruits, seeds, flowers, roots, leaves and/or barks of the above plants or from the whole plants (algae, seaweed).

As contemplated herein, the plant extracts can be used both in pure and diluted form. If they are used in diluted form, they usually contain approx. 2-80% by weight of active substance and as solvent the extracting agent or extracting agent mixture used in their extraction. Suitable extraction agents are usually water and/or alcohols.

The plant extract(s) may be used in the hair care compositions as contemplated herein preferably in total amounts of from about 0.001 to about 1 wt. %, preferably from about 0.05 to about 0.75 wt. % and in particular from about 0.1 to about 0.5 wt. %, the amounts being based on the weight of the hair care composition.

Suitable protein hydrolysates are product mixtures that can be obtained by acidic, basic or enzymatic catalyzed degradation of proteins.

Protein hydrolysates of vegetable, animal and/or marine origin can be used.

Animal protein hydrolysates include elastin, collagen, keratin, silk and milk protein hydrolysates, which may also be in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolysates of vegetable origin are preferred, e.g. soy, almond, rice, pea, potato and wheat protein hydrolysates. Such products are available, for example, under the trademarks Gluadin® (Cognis), DiaMin® (Diamalt), Lexein® (Inolex) and Crotein® (Croda). Cationized protein hydrolysates can also be used, whereby the underlying protein hydrolysate can originate from animals, for example from collagen, milk or keratin, from plants, for example from wheat, corn, rice, potatoes, soy or almonds, from marine life forms, for example from fish collagen or algae, or from biotechnologically obtained protein hydrolysates. The protein hydrolysates underlying the cationic derivatives can be obtained from the corresponding proteins by chemical, especially alkaline or acid hydrolysis, by enzymatic hydrolysis and/or a combination of both types of hydrolysis. The hydrolysis of proteins usually results in a protein hydrolysate with a molecular weight distribution of from about 100 Daltons up to several thousand Daltons. Preferred are those cationic protein hydrolysates whose underlying protein fraction has a molecular weight of 100 to 25000 Dalton, preferably 250 to 5000 Dalton. Furthermore, cationic protein hydrolysates are quaternized amino acids and mixtures thereof. Quaternization of the protein hydrolysates or amino acids is often carried out using quaternary ammonium salts such as N,N-dimethyl-N-(n-alkyl)-N-(2-hydroxy-3-chloro-n-propyl)-ammonium halides. Furthermore, the cationic protein hydrolysates can also be further derivatized. Typical examples of the cationic protein hydrolysates and derivatives are the products known under the INCI designations and commercially available: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The proportion by weight of the protein hydrolysate(s) in the total weight of the hair care composition is preferably from about 0.01 to about 5 wt. %, more preferably from about 0.025 to about 3 wt. % and in particular about 0.05 to about 2 wt. %.

It should have become clear from the foregoing detailed description that the present disclosure may be one of the following:
1. Hair care composition, comprising:
   at least one polyhydric alcohol,
   at least one polyethylene glycol,
   at least one monohydric alcohol,
   at least one quaternary ammonium salt,
2. Hair care composition according to item 1, wherein the at least one polyhydric alcohol is a C3 or C4 alcohol or a mixture thereof
3. A hair care composition according to any of the preceding points, wherein the at least one polyhydric alcohol is glycerol, propanediol or a mixture thereof
4. A hair care composition according to any of the preceding points, wherein the at least one polyhydric alcohol is a mixture of glycerol and propanediol.
5. A hair care composition according to any one of the preceding points, wherein the at least one polyhydric alcohol is present in a concentration of from about 15 wt. % to about 45 wt. % based on the total weight of the hair care composition therein.
6. A hair care composition according to any one of the preceding points, wherein the at least one polyhydric alcohol is present therein in a concentration of from about 21 wt. % to about 42 wt. % based on the total weight of the hair care composition.
7. A hair care composition according to any one of the preceding points, wherein the at least one polyhydric alcohol is present therein in a concentration of from about 25 wt. % to about 40 wt. % based on the total weight of the hair care composition.
8. A hair care composition according to any of the preceding points, wherein the at least one polyhydric alcohol is a mixture of glycerol and propanediol and is present in the hair care composition in a concentration of from about 25 wt. % to about 40 wt. %, based in each case on the total weight of the hair care composition.
9. A hair care composition according to any one of the preceding points, wherein the at least one polyethylene glycol is a polyethylene glycol having a molecular weight of 200 to 600.
10. A hair care composition according to any of the preceding points, wherein the at least one polyethylene glycol is the polyethylene glycol known under the INCI designation PEG-8 having a molecular weight of about 400.
11. A hair care composition according to any one of the preceding points, wherein the at least one polyethylene glycol is present in a concentration of from about 15 wt. % to about 45 wt. % based on the total weight of the hair care composition therein.
12. A hair care composition according to any one of the preceding points, wherein the at least one polyethylene glycol is present in a concentration of from about 21 wt. % to about 42 wt. % based on the total weight of the hair care composition therein.
13. A hair care composition according to any one of the preceding points, wherein the at least one polyethylene glycol is present in a concentration of from about 25 wt. % to about 40 wt. % based on the total weight of the hair care composition therein.
14. A hair care composition according to one of the preceding points, wherein the at least one polyethylene glycol is the polyethylene glycol known under the INCI designation PEG-8 having a molecular weight of about 400 and is present in a concentration of from about 25 wt. % to about 40 wt. %, based in each case on the total weight of the hair care composition.
15. A hair care composition according to any of the preceding points, wherein the at least one monohydric alcohol is ethanol, propanol, butanol or a mixture thereof
16. A hair care composition according to any of the preceding points, wherein the at least one monohydric alcohol is ethanol, propanol or a mixture thereof
17. A hair care composition according to any of the preceding points, wherein the at least one monohydric alcohol is ethanol.
18. A hair care composition according to any one of the preceding points, wherein the at least one monohydric alcohol is present in a concentration of from about 15 wt. % to about 45 wt. % based on the total weight of the hair care composition therein.
19. A hair care composition according to any one of the preceding points, wherein the at least one monohydric alcohol is present in a concentration of from about 21 wt. % to about 42 wt. % based on the total weight of the hair care composition therein.
20. A hair care composition according to any one of the preceding points, wherein the at least one monohydric alcohol is present in a concentration of from about 25 wt. % to about 40 wt. % based on the total weight of the hair care composition therein.
21. A hair care composition according to any of the preceding points, wherein the at least one monohydric alcohol is ethanol and is present in a concentration of from 25 wt. % to 40 wt. %, each based on the total weight of the hair care composition.
22. A hair care composition according to any of the preceding points, wherein the at least one quaternary ammonium salt is an alkyltrimethylammonium salt.
23. A hair care composition according to any of the preceding points, wherein the at least one quaternary ammonium salt is a C14 to C18 alkyltrimethylammonium salt.
24. Hair care composition according to any of the preceding points, wherein the at least one quaternary ammonium salt is hexadecyltrimethylammonium chloride, also known as cetrimonium chloride.
25. A hair care composition according to any one of the preceding points, wherein the at least one quaternary ammonium salt is present in a concentration of from about 0.1 wt. % to about 12 wt. % based on the total weight of the hair care composition therein.
26. A hair care composition according to any one of the preceding points, wherein the at least one quaternary ammonium salt is present in a concentration of from about 0.7 wt. % to about 10 wt. % based on the total weight of the hair care composition therein.
27. A hair care composition according to any one of the preceding points, wherein the at least one quaternary ammonium salt is present in a concentration of from about 1 wt. % to about 9 wt. % based on the total weight of the hair care composition therein.
28. A hair care composition according to any of the preceding points, wherein the at least one quaternary ammonium salt is hexadecyltrimethylammonium chloride, also known as cetrimonium chloride, and is present in a concentration of from about 1 wt. % to about 9 wt. %, each based on the total weight of the hair care composition.
29. A hair care composition according to any one of the preceding points, further comprising water present therein in a concentration of less than about 20 wt. %, preferably less than about 15 wt. %, and more preferably less than about 10 wt. %, each based on the total weight of the hair care composition.
30. A hair care composition according to any one of the preceding items, further comprising water present therein in a concentration of less than about 10 wt. % based on the total weight of the hair care composition.
31. A hair care composition according to any of the preceding points, wherein it is clear and transparent.
32. A hair care composition according to one of the preceding points, wherein its viscosity is at most about 500 mPas, preferably at most about 350 mPas and in particular preferably at most about 200 mPas, measured with a rotational viscometer, model VT 550, of the Haake company, as described in the instructions accompanying this measuring instrument.
33. A hair care composition according to any of the preceding points, wherein it is free of silicones.
34. A hair care composition according to one of the preceding points, wherein it comprises, as the at least one polyhydric alcohol, the at least one polyethylene glycol, the at least one monohydric alcohol and the at least one quaternary ammonium salt, glycerol, the polyethylene glycol known under the INCI designation PEG-8, ethanol and hexadecyltrimethylammonium chloride.
35. A hair care composition according to one of the preceding points, wherein it comprises, as the at least one polyhydric alcohol, the at least one polyethylene glycol, the at least one monohydric alcohol and the at least one quaternary ammonium salt, glycerol, the polyethylene glycol known under the INCI designation PEG-8, ethanol and hexadecyltrimethylammonium chloride.
36. A hair care composition according to any of the preceding points, wherein it comprises from about 25 wt. % to about 35 wt. % of the at least one polyhydric alcohol, from about 25 wt. % to about 35 wt. % of the at least one polyethylene glycol, from about 25 wt. % to about 35 wt. % of the at least one monohydric alcohol, and from about 6 wt. % to about 10 wt. % of the at least one quaternary ammonium salt, each based on the total weight of the hair care composition.
37. Method comprising the steps:
   bringing the hair care composition according to any of the preceding points into contact with preferably wet or damp hair of a human or animal, in particular long scalp hair of a human,
   optionally work in and/or massage to strengthen the contact with the hair,
   optionally waiting for a certain exposure time in the range of 1 second to 3 minutes, preferably 2 seconds to 1 minute and particularly preferably 5 to 10 seconds, and
   rinsing the hair, preferably with an aqueous liquid, particularly preferably with water, to terminate or at least reduce further exposure to the hair care composition.
38. Method according to item 15, wherein the steps described there as optional are obligatory.
39. Use of the hair care composition according to any of the preceding points for caring for preferably wet or damp hair of a human or animal, in particular long scalp hair of a human.
40. Use according to item 17, wherein it takes place on wet hair of a human being.

### Example and Comparison Test

An exemplary hair care product of the present disclosure has the following formulation.

| Substance/precursor used | Concentration [wt. %] |
|---|---|
| Glycerin 99.5% (0.5% H₂0) | 30.25 |
| Polyethylene glycol MG 400 | 30.00 |
| Ethanol 96% denat. (t-Butanol, Bitrex) | 30.00 |
| Dehyquart A CA | 8.00 |
| Croquat WKPS-LQ-(WD) | 0.20 |
| Peg-7 glyceryl cocoate | 0.50 |
| Apricot kernel oil | 0.05 |
| Perfume | 0.50 |
| Nutrilan Keratin W PP | 0.20 |
| Fruitliquid Cranberry B | 0.20 |
| D.S.H C N6 | 0.10 |

Insofar as these are not generally known substances or the INCI designations of such, commercially available precursors were used, which are described below.

Dehyquart A CA is a product comprising water, citric acid and cetrimonium chloride in that order in the weight ratio 74.5:0.5:25. The product Croquat WKPS-LQ-(WD) is a steardimonium hydroxypropyl hydrolyzed collagen in aqueous solution. The product Nutrilan Keratin W PP is water, 2-phenoxyethanol, hydrolyzed keratin and potassium sorbate in this order in the weight ratio 79:0.8:20:0.2. The product Fruitliquid Cranberry is a cranberry extract in water and glycerin. The product D.S.H C N6 is dimethylsilanol hyaluronate in water.

These and other sophisticated formulations were applied to the hair of test subjects and in the test salon in trials on 6 subjects with severely damaged hair, performed in half-side comparison against a very nourishing emulsion-based crucible treatment, significant improvement was achieved in the following parameters (English terms commonly used in the technical field are bracketed for completeness):
Dissemination capability (spreadability)
Distribution (distribution)
Feeling of hair during rinse off (feel of hair during rinse off)
Detangling of wet hair (detangling of wet hair)
Feeling of wet hair (feel of wet hair)
Detangling of dry hair (detangling of dry hair)
Feeling of dry hair (feel of dry hair)
Spring force (bounce)
Suppleness (suppleness)
Lightweight feel (lightweight feel)

It can therefore be readily confirmed that the hair care compositions as contemplated herein achieve a care and repair effect on severely damaged hair that is clearly superior to the established gold standard in this field (very caring emulsion-based crucible treatment), and this not only for a few exotic parameters but practically throughout for all parameters commonly used in the field. This is particularly noteworthy because, contrary to established opinion in the technical field, these results were obtained with a clear single-phase formulation and with the only very short, in some cases negligibly short, exposure times described in detail above.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair care composition, comprising, as a single-phase liquid:
at least one polyhydric alcohol;
at least one polyethylene glycol;
at least one monohydric alcohol; and
at least one quaternary ammonium salt.

2. The hair care composition of claim 1, wherein the at least one polyhydric alcohol comprises a C3 or C4 polyol or a mixture thereof, and is present in a concentration of from about 15 wt. % to about 45 wt. % based on the total weight of the hair care composition.

3. The hair care composition of claim 1, wherein the at least one polyethylene glycol comprises a molecular weight of from about 200 to about 600 and is present in a concentration of from about 15 wt. % to about 45 wt. %, based on the total weight of the hair care composition.

4. The hair care composition of claim 1, wherein the at least one monohydric alcohol is selected from ethanol, propanol, butanol, and combinations thereof, and is present in a concentration of from about 15 wt. % to 45 wt. %, based on the total weight of the hair care composition.

5. The hair care composition of claim 1, wherein the at least one quaternary ammonium salt is further defined as an alkyltrimethylammonium salt and comprises a C14 to C18 alkyltrimethylammonium salt in a concentration of from about 0.1 wt. % to about 12 wt. %, based on the total weight of the hair care composition.

6. The hair care composition of claim 1, further comprising water in a concentration of less than about 10 wt based on the total weight of the hair care composition; and wherein the composition is clear and transparent.

7. The hair care composition of claim 1, wherein:
the at least one polyhydric alcohol is glycerol,
the at least one polyethylene glycol is a polyethylene glycol known under the INCI designation PEG-8,
the at least one monohydric alcohol is ethanol, and
the at least one quaternary ammonium salt is hexadecyltrimethylammonium chloride.

8. The hair care composition of claim 1, comprising:
from about 25% to about 35% by weight of the at least one polyhydric alcohol,
from about 25% to about 35% by weight of the at least one polyethylene glycol,
from about 25% to about 35% by weight of the at least one monohydric alcohol, and
from about 6% to about 10% by weight of the at least one quaternary ammonium salt, each based on the total weight of the hair care composition.

9. A method of treating hair, comprising:
providing the care composition of claim 1;
contacting wet or moist hair of a human for an exposure time of from about 1 second to about 3 minutes; and
rinsing the hair to terminate or reduce further exposure of the hair to the hair care composition.

10. The method of claim 9, wherein the hair is further defined as long scalp hair of a human.

11. The method of claim 9, wherein the exposure time is from about 5 to about 10 seconds.

12. The hair care composition of claim 1, wherein:
the at least one polyhydric alcohol is glycerol, propanediol, or a combination thereof;
the at least one polyethylene glycol is PEG-8;
the at least one monohydric alcohol is ethanol, propanol, or a combination thereof; and
the at least one quaternary ammonium salt is hexadecyltrimethylammonium chloride.

13. The hair care composition of claim 12, consisting essentially of:
from about 25% to about 40% by weight of the at least one polyhydric alcohol;
from about 25% to about 40% by weight of the at least one polyethylene glycol;
from about 25% to about 35% by weight of the at least one monohydric alcohol;
from about 1% to about 9% by weight of the at least one quaternary ammonium salt; and
less than about 10% by weight water, each based on the total weight of the hair care composition.
